(19) United States
(12) Patent Application Publication
(10) Pub. No.: US 2010/0256600 A1
(43) Pub. Date: Oct. 7, 2010

(54) NEUROVASCULAR OTW PTA BALLOON CATHETER AND DELIVERY SYSTEM

(76) Inventor: David A. Ferrera, Redondo Beach, CA (US)

Correspondence Address: Luce, Forward, Hamilton & Scripps LLP, 2050 Main Street, Suite 600, Irvine, CA, 92614, US

(21) Appl. No.: 12/753,812

(22) Filed: Apr. 2, 2010

Related U.S. Application Data
(60) Provisional application No. 61/166,725, filed on Apr. 4, 2009.

(51) Int. Cl. A61B 17/22; A61M 25/10

(52) U.S. Cl. 604/509; 606/200; 604/103.02

(57) ABSTRACT

Briefly stated, a balloon catheter and delivery system, includes in combination a catheter having a proximal end and a distal end, wherein the catheter comprises a delivery lumen, an inflation lumen, and a balloon disposed near the distal end of the catheter; and an expandable device at least partially disposed within the delivery lumen of the catheter, wherein the expandable device comprises a cage-like structure and a tether. An improved method for treating ischemic stroke is likewise disclosed, including crossing an occlusion with the catheter, whereby a channel within the occlusion is created and the balloon is disposed within the channel of the occlusion; inflating the balloon; deflating the balloon; retracting the catheter, whereby the cage-like structure is deployed within the channel of the occlusion; and maintaining a channel of the occlusion with the cage-like structure.

### RELATED APPLICATIONS

This application claims the Paris Convention priority of U.S. Provisional Patent Application Ser. No. 61/166,725, filed Apr. 4, 2009.

### BACKGROUND

This disclosure relates to systems and methods for treating vascular issues. Specifically, this disclosure relates to systems and methods for treating ischemic stroke or other vascular issues caused by an occlusion within blood vessels, above the cerotic artery.

### SUMMARY OF THE DISCLOSURE

Briefly stated, according to embodiments of the present disclosure, a balloon catheter and delivery system is disclosed. The system includes: a catheter having a proximal end and a distal end, wherein the catheter comprises a delivery lumen, an inflation lumen, and a balloon disposed near the distal end of the catheter; and an expandable device at least partially disposed within the delivery lumen of the catheter, wherein the expandable device comprises a cage-like structure and a tether.

According to embodiments of the present disclosure, an improved method for treating ischemic stroke is disclosed. The method may include: providing the balloon catheter and delivery system as disclosed herein to an occlusion; crossing the occlusion with the catheter, whereby a channel within the occlusion is created and the balloon is disposed within the channel of the occlusion; inflating the balloon; deflating the balloon; retracting the catheter, whereby the cage-like structure is deployed within the channel of the occlusion; and maintaining a channel of the occlusion with the cage-like structure.

According to embodiments of the present disclosure, a kit is disclosed. The kit may comprise: a catheter having a proximal end and a distal end, wherein said catheter comprises a delivery lumen, an inflation lumen, and a balloon disposed near said distal end of said catheter; an expandable device at least partially disposed within said delivery lumen of said catheter, wherein said expandable device comprises a cage-like structure and a tether; and directions for use.

### BRIEF DESCRIPTION OF THE FIGURES

The above-mentioned features and objects of the present disclosure will become more apparent with reference to the following description taken in conjunction with the accompanying drawings wherein like reference numerals denote like elements and in which:

FIG. 1 is an illustration of a balloon catheter and delivery system, with a balloon in a deflated state, according to embodiments of the present disclosure;

FIG. 2 is an illustration of a balloon catheter and delivery system, with a balloon in an inflated state, according to embodiments of the present disclosure;

FIG. 3 is an illustration of a balloon catheter and delivery system, with a cage-like structure in a deployed state, according to embodiments of the present disclosure;

FIG. 4 is cross-sectional view of a balloon catheter and delivery system, with a cage-like structure in a retracted state, according to embodiments of the present disclosure;

FIG. 5 is an illustration of a balloon catheter and delivery system shown approaching an occlusion, according to embodiments of the present disclosure;

FIG. 6 is an illustration of a balloon catheter and delivery system shown crossing an occlusion, according to embodiments of the present disclosure;

FIG. 7 is an illustration of a balloon catheter and delivery system, shown with a balloon in an inflated state, according to embodiments of the present disclosure;

FIG. 8 is an illustration of a balloon catheter and delivery system, shown with a balloon in a deflated state after an inflated state, according to embodiments of the present disclosure;

FIG. 9 is an illustration of a balloon catheter and delivery system shown withdrawing from an occlusion and with a cage-like structure in a partially deployed state, according to embodiments of the present disclosure;

FIG. 10 is an illustration of a balloon catheter and delivery system shown withdrawing from an occlusion and with a cage-like structure in a fully deployed state, according to embodiments of the present disclosure; and

FIG. 11 is an illustration of a balloon catheter and delivery system shown fully withdrawn and with a cage-like structure in a temporary or long-term steady-state fully deployed state, according to embodiments of the present disclosure.

### DETAILED DESCRIPTION OF THE DISCLOSURE

The present inventor has discovered that “over the wire” (“OTW”) systems suited to usage in the peripheral vascular and cardio-vascular spaces can be used in the brain and its supporting vasculature.

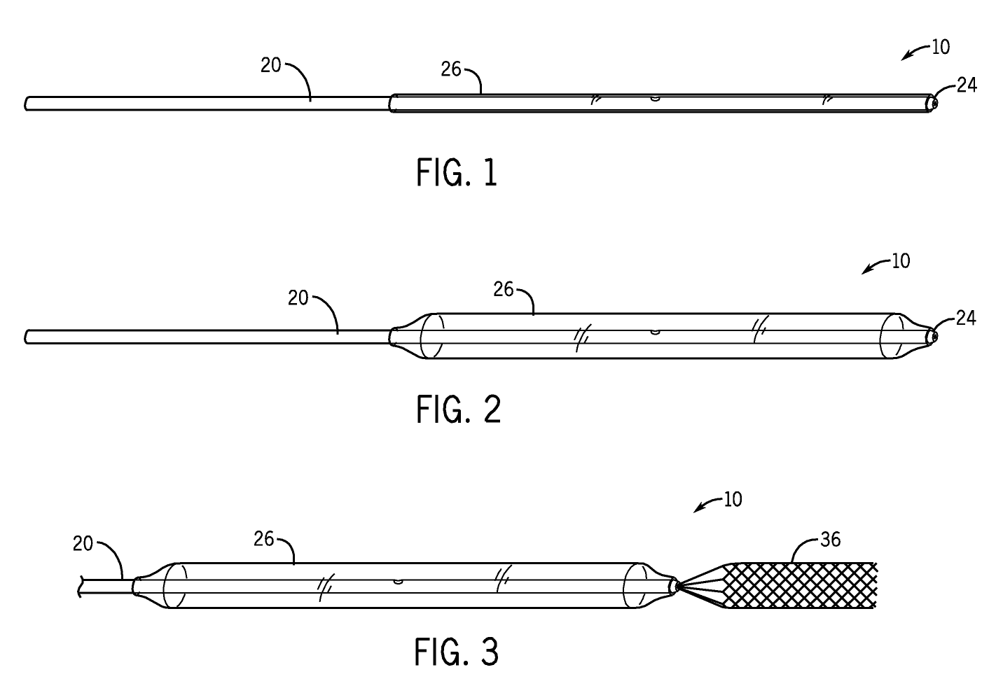

According to embodiments of the present disclosure, a device and method are disclosed for treating occlusions of blood vessels, veins, and arteries, including neurovasculature, such as above the carotid artery. Occlusions may be partial or complete, and may be attributable to one or more of emboli, thrombi, calcified lesions, atheroma, macrophages, lipoproteins, any other accumulated vascular materials, or stenosis. According to embodiments, the systems and methods of the present disclosure facilitate lysis of such occlusions. Expressly incorporated herein are WO2007/089897A3; EP1437097A1, EP0321912A1; U.S. Pat. No. 6,383,205B1, U.S. Pat. No. 5,795,322A U.S. Pat. No. 5,456,667A; U.S. Pat. No. 4,650,466-A. With reference to FIGS. 1-3, according to embodiments of the present disclosure, a balloon catheter and delivery system 10 may include a catheter 20 and a balloon 26. The system 10 may have a distal end 24 and a proximal end 22 (not shown).

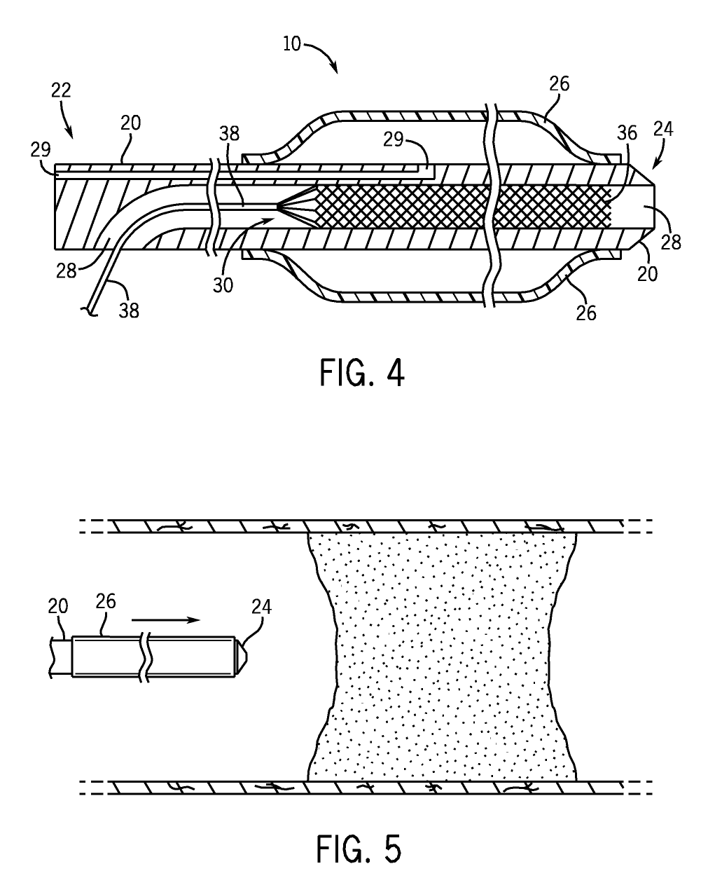

With reference to FIG. 4, according to embodiments of the present disclosure, a balloon catheter and delivery system 10 may comprise a proximal end 22, a distal end 24 and at least one lumen. A catheter 20 may be of any length for performance of minimally invasive vascular treatments. For example, for treatment of stroke, aneurysm, or other treatments within the brain of a patient, a catheter 20 may have a length of between about 135 cm and about 150 cm.

The catheter 10 may be of variable stiffness that is able to track to and through the tortuous anatomy or the cerebral vasculature (i.e., internal carotid artery, MCA, ACA, vertebral and basilar). The catheter 10 may be one or two pieces and may have greater proximal pushability (stiffness) and greater distal flexibility (softness) to allow tracking to distal cerebral arteries.

According to embodiments, there may be provided at least one balloon 26 near a distal end 24 of a catheter 20 for lumen dilatation, treatment of ICAD, vasospasm, flow arrest and remodeling of aneurysm necks during embolization coiling. According to embodiments, a balloon 26 may be disposed outside the outer surface of catheter 20, such that the catheter is concentrically disposed within a portion of balloon 26, and such that balloon 26 expands radially away from catheter 20. The balloon 26 may be a percutaneous transluminal angioplasty (“PTA”) balloon. Such balloons are known in the art. According to embodiments, a plurality of balloons 26 may be provided on an outer surface of catheter 20. According to embodiments, a balloon 26 may have a diameter in an inflated state of between about 0.018″ and about 0.035″.

A balloon 26 may be comprised of materials such as Pebax, nylon, PTFE, polyethylene terephthalate (“PET”), polyurethane, polyester, an elastomeric material, or other suitable materials or mixtures thereof. A balloon 26 may be of any length that facilitates adequate crossing of an occlusion. For example, a balloon 26 may be between about 1.5 cm and about 4.0 cm in length.

According to embodiments, at least one inflation lumen 29 may provide fluid communication to the balloon 26 from the proximal end 22 of the catheter 20. An inflation lumen 29 may provide a fluid to the inner portion of the balloon 26, such that the fluid fills and inflates the balloon 26. The inflation lumen 29 may be open at or near proximal end 22 of the catheter 20, and may be configured to interface with a luer adaptor, fitting, handle, syringe, injector, plunger, or any other one or more selectable items for operation of the balloon catheter and delivery system by a user. Likewise, using ePTFE, PRFE, or other known and lubricious and/or drug eluting elements with the lumens 28 and/or 29 is contemplated.

According to embodiments, an expandable device 30 may be configured to be disposable within the delivery lumen 28. The expandable device 30 may include a tether 38 and a cage-like structure 36. Tether 38 may be attached to the cage-like structure 36 and may be selectively detachable. Tether 38 may extend to or beyond the proximal end 22 of catheter 20. The expandable device 30 may be disposable and trackable within the delivery lumen 28 of the catheter 20.

According to embodiments, at least a portion of a cage-like structure 36 may be tapered at or near a point of attachment with a tether 38. For example, a design may be provided tapering from the diameter of the tether 38 to the largest diameter of the cage-like structure 36. Likewise, alternate geometric configurations for this aspect of the instant teachings are contemplated to be within the scope of the present disclosure: everted, scalloped, and other variant ends, edges are prototyped and being used.

An example of an expandable device 30 may be the IRIIS™ brand system for restoring blood flow in a thrombotic neurovascular occlusion in patients experiencing an ischemic stroke, by Mindframe®, Incorporated (San Clemente, Calif.).

According to embodiments, a cage-like structure 36 may be made of Nitinol to allow it to be compressed and loaded into an introducer for packaging. Similarly, “super-elastic” materials and memory-based materials likewise function, in accordance with the instant systems. According to embodiments, the cage-like structure 36 may be compressible and expandable, such that it may maintain a compressed state when within a lumen and may maintain an expanded state when outside the lumen. According to embodiments, the cage-like structure 36 may be “self-expanding”, such that it expands once unsheathed from the delivery lumen 28 of the catheter 20.

According to embodiments, by attaching it to a delivery wire, the cage-like structure 36 can be placed, retracted, repositioned and recaptured into a catheter. These features allow for the following: 1) perfusion of blood through artery during coiling; 2) perfusion from coiling herniation or prolapse; and 3) removal of the device, mitigating the use of Aspirin and Plavix. The Jervis patents are well know and likewise expressly incorporated herein by reference, including U.S. Pat. No. 6,306,141.

A technical basis for the term “super-elastic” is found in the class of nickel-titanium alloys known as “nitinol” alloys discovered by the United States Navy Ordinance Laboratory. These materials are discussed in length in U.S. Pat. No. 3,174,851 to Beuhler, et al; U.S. Pat. No. 3,351,463 to Rozner, et al; and U.S. Pat. No. 3,753,700 to Harrison, et al. Alloys known to be suitable for this invention are those containing at least 1.5% (wt) and up to about 85% (wt) or more, of one or more alloying members selected from the group consisting of vanadium, chromium, manganese, iron, and cobalt.

According to embodiments, delivery lumen 28 may have an inner diameter to accommodate the cage-like structure 36. According to embodiments, at least one delivery lumen 28 may provide a pathway through the catheter 20 from about the proximal end 22 of the catheter 20 to about the distal end 24 of the catheter 20. A delivery lumen 28 may be open at or near proximal end 22 of the catheter 20, and may be configured to interface with a luer adaptor, fitting, handle, syringe, injector, plunger, or any other one or more selectable items for operation of the balloon catheter and delivery system by a user. As discuss, PTFE, ePTFE and other lubricious and/or eluting elements are incorporated within at least the lumen 28.

According to embodiments, delivery lumen 28 be lined with polytetrafluoroethylene (“PTFE”) or a polymer thereof, alone or in combination with other materials, coatings, coverings, or delivery surfaces or substrates.

According to embodiments, the catheter 20 and the expandable device 30 may be configured to travel together, such that the expandable device 30 may selectively accompany the catheter 20 as the catheter 20 travels through or is emplaced within a vasculature. For example, the catheter 20 and the expandable device 30 may be jointly delivered to a location while the cage-like structure 36 remains within delivery lumen 28.

According to embodiments, the catheter 20 and the expandable device 30 may be configured to be separately disposable, such that they may be moved relative to each other. For example, the expandable device 30 may be advanced or retracted relative to the catheter 20 by advancement or retraction of only the tether 38 at the proximal end 22 of the catheter 20. Likewise, the catheter 20 may be advanced or retracted relative to the expandable device 30 by advancement or retraction of only the catheter 20.

According to embodiments, catheter 20 may be configured to provide tracking over a guide-wire (not shown). One or more lumens of catheter 20 may provide a pathway for a guide-wire using an over-the-wire (OTW) system.

According to embodiments, a method is disclosed for treatment of a vascular occlusion, particularly a neurovascular occlusion. With reference to FIG. 5, according to embodiments of the present disclosure, a balloon catheter and delivery system 10 may be provided to an occlusion.

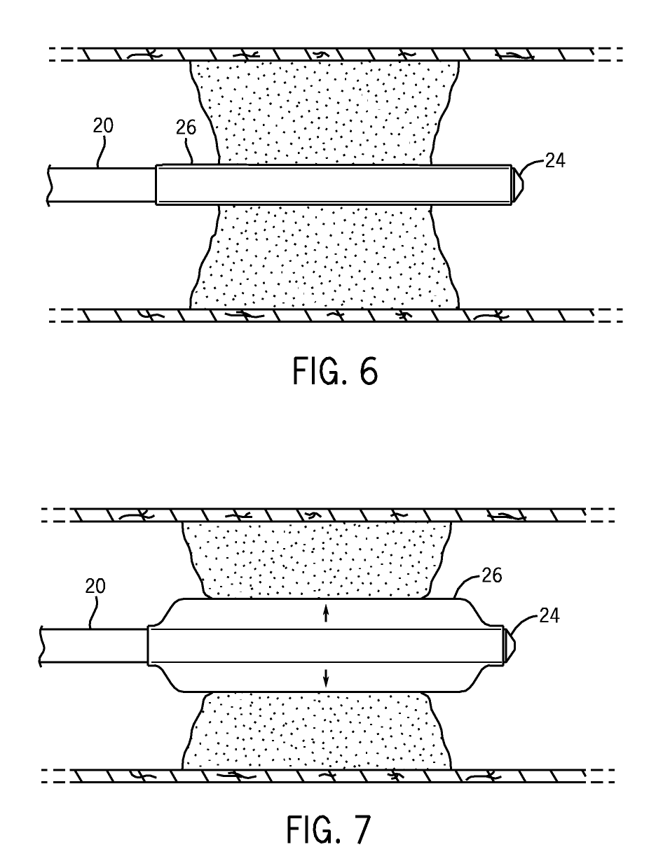

With reference to FIG. 6, according to embodiments of the present disclosure, a balloon catheter and delivery system 10 may cross the occlusion by leading with distal end 24 of catheter 20. Crossing may be effectuated by pressure, force, ablation, or application of one of various types of energy at the distal end 24 of the catheter 20. Crossing may create an initial channel by displacement of the occlusion in the presence of the balloon catheter and delivery system 10.

With reference to FIG. 7, according to embodiments of the present disclosure, a balloon 26 may be inflated or a catheter 20 may otherwise be dilated. Inflation of balloon 26 may further displace or compress at least a portion of the occlusion away from the catheter 20. Thereby, a broader channel may be created by balloon 26, wherein the diameter or cross sectional area of the channel exceeds the diameter or cross sectional area of the catheter 20.

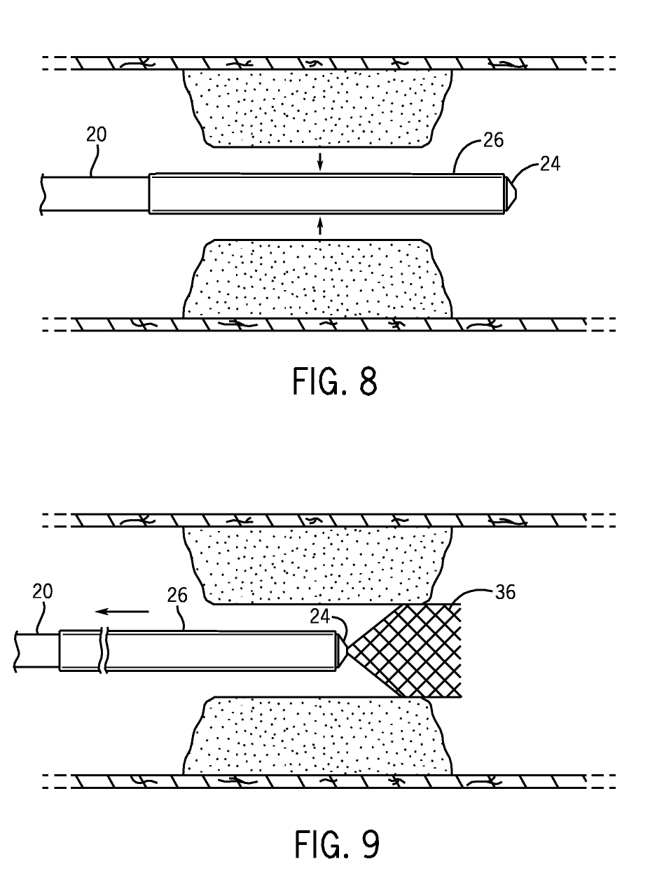

With reference to FIG. 8, according to embodiments of the present disclosure, the balloon 26 may be deflated, whereby the broader channel exceeding the size of the catheter 20 remains open at least temporarily.

With reference to FIG. 9, according to embodiments of the present disclosure, the catheter 20 may be withdrawn from an occlusion. The operation of withdrawing the catheter 20 may simultaneously result in unsheathing and deployment of the cage-like structure 36. Deployment of the cage-like structure 36 may result in an expansion of any portion of the cage-like structure 36 that is not within the lumen 28 of the catheter 20.

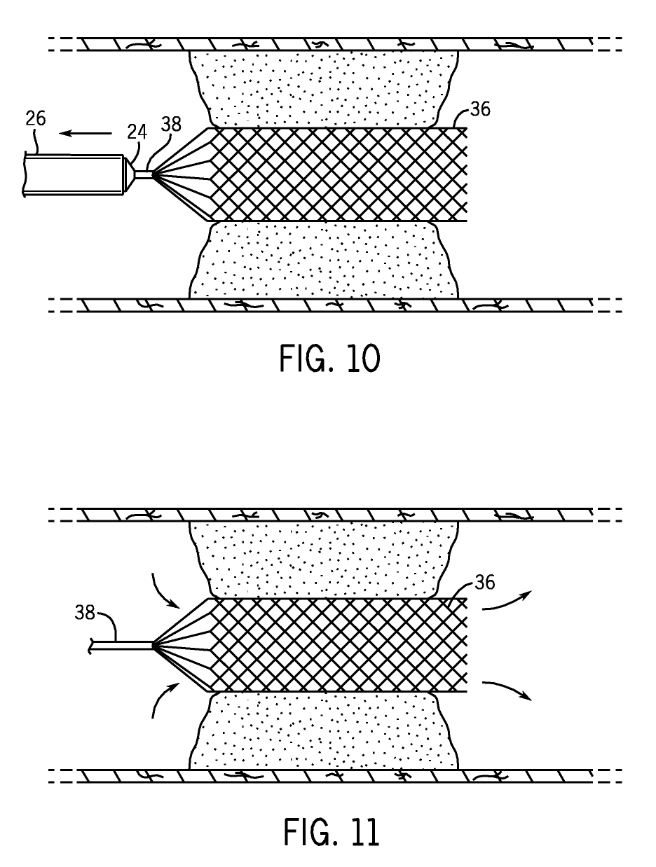

With reference to FIG. 10, according to embodiments of the present disclosure, the catheter 20 may be withdrawn such that the cage-like structure 36 may achieve a fully deployed state. For example, a fully deployed state may be achieved when the entire length of the cage-like structure 36 is outside the delivery lumen 28 of catheter 20, or when at least a length of the cage-like structure 36 corresponding to the length of the occlusion is outside the delivery lumen 28 of catheter 20. Expansion of the cage-like structure 36 may maintain the approximate size and dimensions of the broader channel created by previously inflating balloon 26.

With reference to FIG. 11, according to embodiments of the present disclosure, the cage-like structure 36 may achieve a temporary or long-term steady-state fully deployed state, wherein improved flow may be achieved through the occlusion. The flow through the channel may facilitate lysis of the occlusion and its constituent parts. The cage-like structure 36 may maintain the channel created by the dilation or inflation of the balloon 26, even as the channel deforms or is otherwise modified by the improved flow. According to embodiments, the cage-like structure 36 may be maintained within the channel of the occlusion.

According to embodiments, the cage-like structure 36 may be retracted into the delivery lumen 28 of the catheter 20, and the catheter 20 may be removed from the location of the occlusion.

While the method and agent have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. A neurovascular treatment method, comprising, in combination:
providing a balloon catheter and delivery system to an occlusion lodged above the cerotic artery, wherein said balloon catheter and delivery system comprises, in combination: a catheter having a proximal end and a distal end, wherein said catheter comprises a delivery lumen, an inflation lumen, and a balloon disposed near said distal end of said catheter; and an expandable device at least partially disposed within said delivery lumen of said catheter, wherein said expandable device comprises a cage-like structure and a tether;
crossing an occlusion with said catheter, whereby a channel within said occlusion is created and said balloon is disposed within said channel of said occlusion;
inflating said balloon;
deflating said balloon;
retracting said catheter, whereby said cage-like structure is deployed within said channel of said occlusion; and
maintaining said channel of said occlusion with said cage-like structure.

2. The method of claim 1, wherein inflating said balloon enlarges said channel of said occlusion.

3. The method of claim 1, wherein said cage-like structure expands upon retraction of said catheter.

4. The method of claim 1, wherein maintaining said channel of said occlusion provides improved vascular flow.

5. The method of claim 1, wherein maintaining said channel of said occlusion facilitates lysis of said occlusion.

6. The method of claim 1, wherein inflating said balloon is effectuated by provided fluid to said balloon through said inflation lumen.

7. The method of claim 1, further comprising advancing said catheter to recapture said cage-like structure.

8. The method of claim 7, wherein the occlusion is lodged in one of the internal cerotic artery, MCA, ACA, vertical or basilar vessels.

9. A balloon catheter and delivery system, comprising, in combination:
a catheter having a proximal end and a distal end, wherein said catheter comprises a delivery lumen, an inflation lumen, and a balloon disposed near said distal end of said catheter; and
an expandable device at least partially disposed within said delivery lumen of said catheter, wherein said expandable device comprises a cage-like structure and a tether.

10. The system of claim 9, wherein said balloon catheter and delivery system is an over-the-wire (OTW) system.

11. The system of claim 9, wherein said balloon is a PTA balloon.

12. The system of claim 9, wherein said inflation lumen is configured to provide fluid communication between said proximal end of said catheter to said balloon.

13. The system of claim 9, wherein said tether is fixedly attached to said cage-like structure.

14. The system of claim 9, wherein said tether extends through said delivery lumen from said cage-like structure to said proximal end of said catheter.

15. The system of claim 9, for use above the carotid artery.

16. The system of claim 9, for use in the brain.

17. A kit, comprising:
a catheter having a proximal end and a distal end, wherein said catheter comprises a delivery lumen, an inflation lumen, and a balloon disposed near said distal end of said catheter;
an expandable device at least partially disposed within said delivery lumen of said catheter, wherein said expandable device comprises a cage-like structure and a tether; and
directions for use.

18. The kit of claim 17, wherein said delivery lumen is coated with a liner selected from the group of lubricious coating consisting of PTFE, PET, polymers and combination there of.

19. The kit of claim 17, wherein said cage-like structure includes at least one of an everted end and a scalloped end near said tether.

20. The kit of claim 17, wherein said cage-like structure is coated with at least one drug to facilitate lysis of an occlusion.
